(19) United States
(12) Patent Application Publication
(10) Pub. No.: US 2022/0249778 A1
(43) Pub. Date: Aug. 11, 2022

(54) SEALING ARRANGEMENT FOR DRUG DELIVERY DEVICE

(71) Applicant: AMGEN INC., Thousand Oaks, CA (US)

(72) Inventors: Mehran Mojarrad, Thousand Oaks, CA (US); Ali Nekouzadeh, Simi Valley, CA (US); Sheldon B. Moberg, Thousand Oaks, CA (US); Scott R. Gibson, Simi Valley, CA (US); Paul Daniel Faucher, Escondido, CA (US)

(21) Appl. No.: 17/627,008

(22) PCT Filed: Jul. 16, 2020
(86) PCT No.: PCT/US20/42225

§ 371 Date: Jan. 13, 2022

Related U.S. Application Data
(60) Provisional application No. 62/875,763, filed on Jul. 18, 2019.

(51) Int. Cl. A61M 5/31; A61M 5/145; A61M 5/162; A61M 5/20; A61M 5/24

(52) U.S. Cl. CPC A61M 5/3129; A61M 5/14526; A61M 5/162; A61M 2005/3131; A61M 5/2046; A61M 5/2459; A61M 5/2053

(57) ABSTRACT

A drug delivery device includes a housing releasably coupled with a patient, a container disposed in the housing, a plunger disposed in the container, an activation mechanism, and a seal member. The container includes a wall with an interior surface. The plunger includes an interior surface and is moveably disposed in the container. The interior surfaces of the wall and the plunger define a reservoir adapted to contain a drug. The activation mechanism is adapted to release a drive fluid for moving the plunger through the container to expel the drug therefrom. The seal member is moveably disposed in the container and is adapted to inhibit ingress of the drive fluid into the reservoir.

### CROSS-REFERENCE TO RELATED APPLICATION

This application claims the benefit of U.S. Provisional Application No. 62/875,763, filed on Jul. 18, 2019, which is hereby incorporated by reference herein in its entirety.

### FIELD OF DISCLOSURE

The present disclosure generally relates to drug delivery devices and, more particularly, sealing various components within a drug delivery device driven by a drive fluid so as prevent leaks of the drive fluid.

### BACKGROUND

Drug delivery devices, such as injectors, are used to deliver liquid drugs to a patient. Upon activation, a drug delivery device may expel a drug stored within an internal reservoir of a primary container through a needle, cannula, or other delivery member into the patient.

High viscosity drugs, including some biologics, can require significant force to push the drug through the delivery member into the patient. Certain conventional drive systems are unable to provide the requisite force needed for injecting high viscosity drugs, or are unable to do so within a preferred timeframe. Drive systems powered by a pressurized fluid can be sufficiently powerful for high viscosity drugs but introduce a risk that the pressurized fluid will breach the sealed reservoir of the primary container and interact with the drug. This may affect the composition of the drug and, in certain instances, compromise its integrity and/or render it unsafe for patient administration.

As described in more detail below, the present disclosure sets forth systems for delivery devices embodying advantageous alternatives to existing systems and methods, and that may address one or more of the challenges or needs mentioned herein, as well as provide other benefits and advantages.

### SUMMARY

In accordance with a first aspect, a drug delivery device includes a housing releasably coupled with a patient, a container disposed in the housing, a plunger disposed in the container, an activation mechanism, and a seal member. The container includes a wall with an interior surface. The plunger includes an interior surface and is moveably disposed in the container. The interior surfaces of the wall and the plunger define a reservoir adapted to contain a drug. The activation mechanism is adapted to release a drive fluid for moving the plunger through the container to expel the drug therefrom. The seal member is moveably disposed in the container and is adapted to inhibit ingress of the drive fluid into the reservoir. Upon engaging the activation mechanism, the drive fluid contacts the seal member, causing the plunger to move through the container to expel the drug from the container. In some examples, the drive fluid may be in the form of a pressurized liquid and/or a pressurized gas, though other types of drive fluids may be used.

In some approaches, the seal member is adapted to expand upon the release of the drive fluid. The seal member may include a first portion that is fixedly coupled with the plunger, and may further include a second portion that is fixedly coupled with the interior surface of the wall of the container. In some examples, the seal member is in the form of a telescoping bellows. The telescoping bellows is operably coupled to the plunger. In some examples, the seal member is in the form of an accordion bellows. The accordion bellows is operably coupled to the plunger.

In some examples, the seal member is in the form of a platform member and a flexible membrane operably coupled to the platform member. The platform member has an outer surface defining a rolling gap with the interior surface of the wall of the container. The flexible membrane has a storage state where the flexible membrane is folded at least partially within the rolling gap and an operative state where the drive fluid causes the flexible membrane to unroll and slide against at least one of the platform member or the interior surface of the wall of the container. In some examples, a retaining cup may be provided that is disposed about the platform member for guiding the flexible membrane in the operative state.

In some examples, the drug delivery device further includes a plug covering an opening formed in the wall of the container. The plug may include a drive fluid channel to allow the drive fluid to flow therethrough upon being released. The plug may further include a coupling region that accommodates a portion of the seal member. In some forms, the coupling region includes a groove to accommodate a portion of the seal member and an O-ring.

In some approaches, the drug delivery device further includes a container access mechanism at least partially disposed within the housing and being operably coupled to the container. The container access mechanism includes a needle or a cannula and a sterile barrier disposed proximal to the needle or cannula in a first configuration where the sterile barrier is intact. In some of these examples, upon engaging the activation mechanism, relative movement between the needle or cannula and the sterile barrier causes the needle or cannula to pierce the sterile barrier to allow the medicament to be expelled from the container.

In some examples, the drug delivery device may also include an insertion mechanism in fluid communication with the container. The insertion mechanism includes a needle or a cannula to be inserted into the user to deliver the medicament.

In accordance with a second aspect, an assembly for a drug delivery device includes a container, a plunger disposed in the container, an activation mechanism, and a seal member. The container includes a wall with an interior surface. The plunger has an interior surface and is moveably disposed in the container. The interior surface of the wall and the interior surface of the plunger define a reservoir adapted to contain a drug. The activation mechanism is adapted to release a drive fluid for moving the plunger through the container to expel the drug from the container. The seal member is moveably disposed in the container and is adapted to inhibit ingress of the drive fluid into the reservoir.

### BRIEF DESCRIPTION OF THE DRAWINGS

The above needs are at least partially met through provision of the sealing arrangement for a drug delivery device described in the following detailed description, particularly when studied in conjunction with the drawings, wherein:

The accompanying figures show embodiments according to the disclosure and are exemplary rather than limiting.

FIG. 1 illustrates a schematic representation of an example arrangement of a drug delivery device having a sealing arrangement in accordance with various embodiments;

FIG. 2A illustrates a first example seal member for the example drug delivery device of FIG. 1 in the form of a telescoping bellows member in accordance with various embodiments;

FIG. 2B illustrates the example seal member of FIG. 2A upon initiating drug delivery in accordance with various embodiments;

FIG. 3A illustrates a second example seal member for the example drug delivery device of FIG. 1 in the form of an accordion bellows member in accordance with various embodiments;

FIG. 3B illustrates the example seal member of FIG. 3A upon initiating drug delivery in accordance with various embodiments;

FIG. 4A illustrates a third example seal member for the example drug delivery device of FIG. 1 in the form of a platform and flexible membrane arrangement in accordance with various embodiments;

FIGS. 4B-4E illustrate the example seal member of FIG. 4A during an operational state of the drug delivery device in accordance with various embodiments;

FIG. 5A illustrates a fourth example seal member for the example drug delivery device of FIG. 1 in the form of a platform, a flexible membrane, and retaining cup arrangement in accordance with various embodiments;

FIGS. 5B-5D illustrate the example seal member of FIG. 5A during an operational state of the drug delivery device in accordance with various embodiments;

FIGS. 6A and 6B illustrate a fifth example seal member for the example drug delivery device of FIG. 1 during an operational state of the drug delivery device in accordance with various embodiments;

FIG. 7A illustrates a sixth example seal member for the example drug delivery device of FIG. 1 in accordance with various embodiments; and

FIGS. 7B and 7C illustrate the example seal member of FIG. 7A during an operational state of the drug delivery device in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure generally relates to a drive system for a drug delivery device that includes a sealing arrangement to prevent leaking of a drive fluid. The drug delivery device may include a housing defining a shell and an inner volume, a drive system, a container including a reservoir filled or adapted to be filled with a drug, an insertion mechanism, an activation mechanism, and a fluid flow connection, each of which is at least partially disposed within the housing. The drive system is driven via a drive fluid (e.g., a pressurized gas and/or a pressurized liquid) that when released exerts a force to expel the drug from the container. A seal member is disposed between the drive fluid and a plunger included in the container and acts to inhibit ingress of the drive fluid into the reservoir and/or other components for which exposure to the drive fluid is not desirable. In some examples, this seal member is additionally adapted to expand upon the release of the drive fluid.

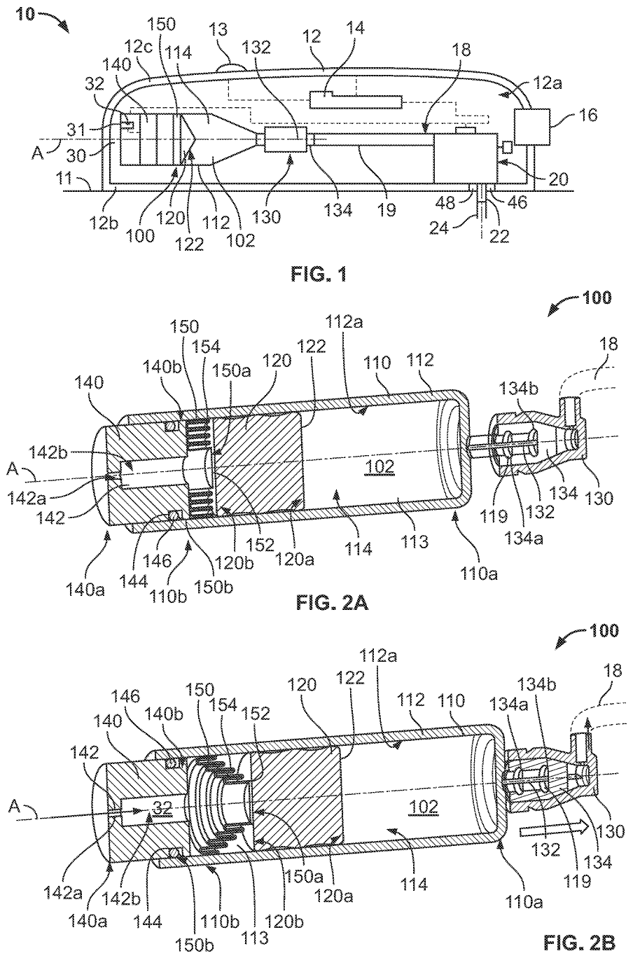

Referring to the Figures, a general drug delivery device 10 is provided that may include any number of aspects of the sealing arrangement herein described. In some embodiments, including the one illustrated in FIG. 1, the drug delivery device 10 may be configured as a wearable drug delivery device, such as an on-body injector or an ambulatory infusion pump, that may be releasably coupled with a patient (e.g., to a patient's tissue 11 such as the patient's skin) to administer delivery of a drug treatment. In other embodiments, the drug delivery device 10 may be in the form of an autoinjector, a pen injector, or any other type of handheld devices including hybrids thereof. The drug delivery device 10 may be operated to subcutaneously or transdermally deliver a drug to a patient. The drug delivery device 10 may be configured to automatically deliver a fixed or a patient/operator-settable dose of a drug over a controlled or selected period of time. The drug delivery device 10 may be intended for self-administration by the patient, but may also be used by a caregiver or a formally trained healthcare provider to administer an injection.

The drug delivery device 10 has a housing 12 that is releasably coupled with the patient's tissue 11 and that defines a shell and having an inner volume 12a, a needle insertion mechanism 20, an activation mechanism 30, and a sealing arrangement 100, each of which may be at least partially disposed within the housing 12. It is appreciated that the releasable coupling between the housing 12 and the patient's skin 11 can include any coupling or couplings that allow the drug delivery device 10 to be selectively secured to the patient, including the user holding the device 10 against the injection site, a suction force, an adhesive, or other force holding the device 10 to the patient. Further, the drug delivery device may include a controller 14 and an actuator 16 (e.g., a depressible button) that is arranged on an exterior of the housing 12.

Generally, the sealing arrangement 100 includes a container 110 (which, in some examples, may be referred to as a primary container), a plunger 120, a container access mechanism 130, a plug 140, and a seal member 150. The container 110 has a wall 112 that includes an interior surface 112a defining an interior volume 113. The plunger 120 is moveably disposed within the container 110 and has a first end 120a and a second end 120b. The first end 120a of the plunger 120 includes an interior surface 122. The interior surface 112a of the container 110 and the interior surface 122 of the plunger 120 define a reservoir 114 that contains a drug 102.

The housing 12 may include a bottom wall 12b to contact or to be releasably coupled (e.g., adhered with an adhesive) with the patient's skin 11, and a top wall 12c including one or more visual feedback mechanisms 13 such as, for example a window, an opening, and/or an illumination system (not illustrated) for viewing the container 110 and the drug 102 contained therein. The one or more visual feedback mechanisms 13 may be used to communicate information to the user about the operational state of the drug delivery device 10 and/or the condition of the medicament or drug 102. An opening 46 may be formed in the bottom wall 12b, and optionally a pierceable sterile barrier or septum 48 may extend across the opening 46 to seal the interior of the housing 12 prior to use. In some embodiments, the pierceable sterile barrier 48 may be omitted, and instead a removable sealing member (not illustrated) may cover and seal closed the opening 46 prior to use. The exterior of the needle insertion mechanism 20 may be defined by an insertion/retraction mechanism housing that is separate from the housing 12.

A fluid pathway connector 18 connects the sealing arrangement 100, and more specifically the container 110, to the needle insertion mechanism 20. The actuator 16 is configured to initiate operation of the drug delivery device 10 by activating, via mechanical and/or electrical means (shown in dotted lines in FIG. 1), the activation mechanism 30, the needle insertion mechanism 20, the controller 14, and/or other mechanisms and/or electronics. In some examples, wireless communication may be employed to cause the device 10 to be activated. In embodiments where the actuator 16 is a button that is depressed or otherwise physically moved by a user or patient, the actuator 16 may be configured to exert a motive force needed to activate the needle insertion mechanism 20, the fluid pathway connector 18, the activation mechanism 30, the controller 14, and/or other mechanisms. In such embodiments, the actuator 16 may be physically connected to, either directly or indirectly via a mechanical linkage, the needle insertion mechanism 20, the activation mechanism 30, the fluid pathway connector 18, and/or other mechanisms such that manually depressing or otherwise interacting with the actuator 16 supplies the motive force necessary to activate the needle insertion mechanism 20, the activation mechanism 30, the fluid pathway connector 18, and/or other mechanisms.

The fluid pathway connector 18 defines a sterile fluid flow path 19 between the container 110 and the needle insertion mechanism 20. The container access mechanism 130 is coupled to the fluid pathway connector 18 and is configured to insert a container needle 132 through a septum 134 associated with and/or covering the container 110 to establish fluid communication between the container 110 and the sterile fluid flow path 19 in response to activation of the drug delivery device 10, for example, via the actuator 16. In the illustrated examples, relative movement between the container 110 and the container access mechanism 130 causes the container needle 132 to pierce the septum 134. In some examples, the container needle 132 may be staked to the container 110 such that the container needle 132 cannot move relative to the wall 112 of the container 110; whereas, in other examples, the container needle 132 may be moveable relative to the container 110 and may access the reservoir 114 of the container 110 by piercing through a septum or other sterile barrier covering an opening in the container 110 during operation or set up the drug delivery device 10. In some examples, the needle insertion mechanism 20 and the container 110 and/or other components of the sealing arrangement 100 such as the container access mechanism 130 may be integrated into a single unit, and thus the fluid pathway connector 18 may not be included in the drug delivery device 10.

For example, in some embodiments, manually depressing or otherwise moving the actuator 16 may cause the fluid pathway connector 18 and the container access mechanism 130 to move towards the container 110, or cause the container 110 to move towards the fluid pathway connector 18 and the container access mechanism 130, and thereby cause the container needle 132 to penetrate through the seal member or septum 134, thereby creating a fluid flow path between the reservoir 114 and the fluid flow path 19. In the illustrated examples, the container 110 includes a coupling mechanism in the form of an annular protrusion 119 that engages first and second annular grooves 134a, 134b formed in the septum 134. In these examples, prior to drug administration, the annular protrusion 119 is retained in the first annular groove 134a. After or simultaneous with actuation of the actuator 16, relative movement between the container 110 and the container access mechanism 130 causes the annular protrusion 119 to disengage the first annular groove 134a and become inserted into the second annular groove 134b. Accordingly, the container 110 is restrained relative to the container access mechanism 130, and thus the flow path from the reservoir 114 and the fluid flow path 19 is retained.

Additionally, or alternatively, the actuator 16 may operate as an input device that transmits an electrical and/or mechanical signal to the controller 14, which in turn may execute programmable instructions to control operation of the needle insertion mechanism 20, the activation mechanism 30, the fluid pathway connector 18, and/or other mechanisms. In such embodiments, the controller 14 may include a processor (e.g., a microprocessor) and a non-transitory memory for storing the programmable instructions to be executed by the processor. Furthermore, in such embodiments, the drug delivery device 10 may include an internal actuator (e.g., an electric motor, a pneumatic or hydraulic pump, and/or a source of pressurized gas or liquid) which is separate from the actuator 16 and which, in response to an electrical control signal received from the controller 14, exerts the motive force needed to activate the needle insertion mechanism 20, the activation mechanism 30, the fluid pathway connector 18, and/or other mechanisms.

The activation mechanism 30 may include any number of components and/or sub-components to cause a drive fluid 32 to drive, urge, and/or exert a force on the plunger 120 to cause the drug or medicament 102 stored therein to be dispensed therefrom. In some examples, the drive fluid 32 may be a compressed CO₂ gas or other compressed gas and/or a compressed liquid which is initially stored within a pressure vessel or other container 31, and the activation mechanism 30 may be configured to release the compressed gas and/or liquid from the pressure vessel or other container by opening a valve, which allows the compressed gas and/or liquid to flow into the container 110. In other examples, the activation mechanism 30 may be in the form of a hydro-pneumatic actuation system whereby a hydraulic and/or pneumatic force is exerted on the drive fluid 32 to move the plunger 120 through the container 110 to expel the drug 102 therefrom. In other examples, the activation mechanism 30 may include any number of resilient members (e.g., springs) that exert an urging force on the drive fluid 32. Examples of suitable activation mechanisms 30 are described in U.S. App. No. 62/543,058, filed on Aug. 9, 2017, the entire contents of which are incorporated by reference herein. Other examples of suitable activation mechanisms 30 are possible.

The needle insertion mechanism 20 includes a needle 22 and/or a cannula 24 having a storage state where the needle 22 and/or the cannula 24 is retracted within the housing 12 and an operative state wherein a tip of the needle 22 and/or the cannula 24 is deployed through the opening 46. Upon activation of the drug delivery device 10, the drug delivery device 10 may enable, connect, or open necessary connections to establish fluid communication between the container 110 and the fluid pathway connector 18. Simultaneously or subsequently, the needle insertion mechanism 20 may insert the needle 22 into the patient 11. The needle 22 may be constructed of material this is rigid or flexible. In examples where the needle 22 is rigid, the needle 22 may be made of a material that is more rigid and/or harder than the cannula 24. For example, the needle 22 may be made of metal and the cannula 24 may be made of plastic or another polymer. The relative flexibility of the cannula 24 may allow it to be disposed subcutaneously within the patient's tissue 11 for a period of a time without causing pain or discomfort to the patient. In examples where the needle 22 is flexible, the needle 22 may be constructed from a super-elastic material such as nitinol, a polymer, or another material that allows the needle 22 to follow a curved path without sustaining damage. In examples where both the needle 22 and cannula 24 are included, the needle 22 may function as a trocar for creating a pathway through the patient's tissue to facilitate insertion of the cannula 24. Immediately or shortly after the cannula 24 has been inserted, the needle 22 may be retracted back towards the housing 12 leaving the cannula 24 within the patient's tissue for subcutaneous delivery of the drug. In other examples, the cannula 24 may be omitted and the needle 22 may remain within the patient's tissue after insertion for subcutaneous delivery of the drug. In still further examples, the needle 22 may be omitted and the cannula 24, which may be constructed of a non-metal material such as a polymer, may be inserted by itself into the patient's tissue for subcutaneous delivery of the drug. After needle 22 and/or cannula 24 has been inserted, the activation mechanism 30, by releasing or pushing on the drive fluid 32, may force the drug or medicament 102 stored in the container 110 through the sterile fluid flow path 19 of the fluid pathway connector 18 and into the needle insertion mechanism 20 for subcutaneous delivery to the patient 11.

After the bottom wall 12b of the housing 12 is attached to the patient's skin 11, the needle insertion mechanism 20 may be activated to move a delivery member from a retracted position within the housing 12 to a deployed position extending outside of the housing 12. In the present embodiment, this may include the needle insertion mechanism 20 inserting the needle 22 and the cannula 24 through the septum 48 and into the patient's skin 11 and subcutaneous tissue. Immediately or shortly thereafter, the needle insertion mechanism 20 may automatically retract the needle 22, leaving the distal open end of the cannula 24 inside the patient for subcutaneous delivery of the drug 102. The needle 22 may be solid and have a sharpened end for piercing the patient's skin 11, whereas the cannula 24 may be hollow and have a blunt end.

In some embodiments, the needle insertion mechanism 20 may include one or more springs (e.g., coil springs, torsion springs, etc.) initially retained in an energized state, and which are released upon depression of the actuator 16 in order to insert the needle 22 and cannula 24, or hollow needle, into the patient. Furthermore, retraction of the needle 22 may be achieved by the automatic release of another spring after the needle 22 and cannula 24 have been inserted into the patient. Other power sources for insertion and/or retraction are possible, including, for example, an electric motor, a hydraulic or pneumatic pump, or a canister that releases a pressurized gas or pressurized liquid to provide actuation energy.

As previously noted, the container 110 includes a wall having the interior surface 112a that defines the reservoir 114 that is filled with the drug 102, and additionally includes a first end 110a and a second end 110b. In some embodiments, the reservoir 114 may be pre-filled with the drug 102 by a drug manufacturer prior to installation of the container 110 in the drug delivery device 10. In some embodiments, the container 110 may be rigidly connected to the housing 12 such that the container 110 cannot move relative to the housing 12; whereas, in other embodiments, the container 110 may be slidably connected to the housing 12 such that the container 110 can move relative to the housing 12 during operation of the drug delivery device 10. The container 110 may have an elongate, barrel-like or cylindrical shape extending along a longitudinal axis A. In embodiments where the drug delivery device 10 is configured as an on-body injector, the longitudinal axis A of the container 110 may be perpendicular or substantially perpendicular, or otherwise non-parallel, to a direction in which the needle insertion mechanism 20 inserts a delivery member such as the cannula 24 into the patient 11. This configuration may allow the on-body injector to have a generally planar, low-profile shape that can be worn by the patient without impeding the patient's movement. Initially, the plunger 120 may be positioned in the container 110 at or near the second end 110b thereof. The plunger 120 may sealingly and slidably engage the interior surface 112a of the wall 112, and thus is movable relative to the wall 112. Put differently, the plunger 120 acts as a seal that restricts the drug 102 from exiting the second end 110b of the container 110.

The volume of the drug 102 contained in the reservoir 114 prior to delivery may be: any volume in a range between approximately (e.g., ±10%) 0.5-20 mL, or any volume in a range between approximately (e.g., ±10%) 0.5-10 mL, or any volume in a range between approximately (e.g., ±10%) 1-10 mL, or any volume in a range between approximately (e.g., ±10%) 1-8 mL, or any volume in a range between approximately (e.g., ±10%) 1-5 mL, or any volume in a range between approximately (e.g., ±10%) 1-3.5 mL, or any volume in a range between approximately (e.g., ±10%) 1-3 mL, or any volume in a range between approximately (e.g., ±10%) 1-2.5 mL, or any volume in a range between approximately (e.g., ±10%) 1-2 mL, or any volume equal to or less than approximately (e.g., ±10%) 4 mL, or any volume equal to or less than approximately (e.g., ±10%) 3.5 mL, or any volume equal to or less than approximately (e.g., ±10%) 3 mL, or any volume equal to or less than approximately (e.g., ±10%) 2.5 mL, or any volume equal to or less than approximately (e.g., ±10%) 2 mL, or any volume equal to or less than approximately (e.g., ±10%) 1.5 mL, or any volume equal to or less than approximately (e.g., ±10%) 1 mL, or any volume equal to or greater than approximately (e.g., ±10%) 2 mL, or any volume equal to or greater than approximately (e.g., ±10%) 2.5 mL, or any volume equal to or greater than approximately (e.g., ±10%) 3 mL. The reservoir 114 may be completely or partially filled with the drug 102. The drug 102 may be one or more of the drugs listed below under the heading “Drug Information”, such as, for example, a granulocyte colony-stimulating factor (G-CSF), a PCSK9 (Proprotein Convertase Subtilisin/Kexin Type 9) specific antibody, a sclerostin antibody, or a calcitonin gene-related peptide (CGRP) antibody.

In the illustrated examples, the plug 140 is at least partially disposed within the interior volume 113 of the container 110 at the second end 110b thereof. The plug 140 may be constructed from any suitable material including, for example, rubber, metal, a polymer, and the like, or any combination thereof. The plug 140 has a first end 140a and a second end 140b, and additionally includes a channel or bore 142 that extends between the first and second ends 140a, 140b. The plug 140 is operably coupled to the activation mechanism 30 using any number of approaches that allow the drive fluid 32 to enter into the bore 142 at the first end 140a and exit at the second end 140b of the bore 142 and into the interior volume 113 of the container 110. In some examples, the plug 140 is coupled to the activation mechanism 30 via tubing, a nozzle, and/or other similar components.

In the illustrated example, the bore 142 includes a narrow region 142a which may accept the tubing, nozzle, and/or similar component, and further includes a wide region 142b that allows for the drive fluid 32 to properly flow therethrough and enter into the interior volume 113 of the container 110.

The plug 140 additionally includes a coupling region 144 that accommodates a portion of the seal member 150. For example, the coupling region 144 may be in the form of a groove 144 that receives a portion of the seal member 150 and an O-ring 146 or other sealing device.

The seal member 150 is moveably disposed within the interior volume 113 of the container 110. The seal member 150 is constructed from any number of generally fluid-impermeable materials such as, for example pharmaceutically compatible silicone rubber, butyl rubber, butadiene rubber, neoprene, Viton, Buta-N, bromobutyl, chlorobutyl, Flurotec, and/or any other polymers and/or elastomeric materials, and can have additional coatings as lubricant or enhancements to barrier properties. The seal member 150 is positioned between the second end 140b of the plug 140 and the second end 120b of the plunger 120. The seal member 150 includes a first end 150a that is positioned adjacent to and/or is operably coupled with the second end 120b of the plunger 120, and further includes a second end 150b that is positioned adjacent to and/or operably coupled with the second end 140b of the plug 140 as well as the second end 110b of the container 110. Generally, the seal member 150 inhibits and/or prevents ingress of the drive fluid 32 into the reservoir 114, and can additionally inhibit and/or prevent the drive fluid 32 from contacting the plunger 120 and/or certain portions of the container wall 112. Further, the seal member may expand upon the release of the drive fluid 32. The second end 150b of the seal member 150 may be disposed about the second end 140b of the plug 140 and may wrap around an exterior of the O-ring 146 inserted into the groove 144 of the plug 140, and as such, the seal member 150 may completely inhibit ingress of the drive fluid 32 from entering the reservoir 114, from contacting the plunger 120, and/or from contacting the container 110. Put differently, in some examples, the second end 150b of the seal member may be fixedly coupled with the interior surface 112a of the wall 112 of the container 110, while the first end 150a may be fixedly coupled with the second end 120b of the plunger 120.

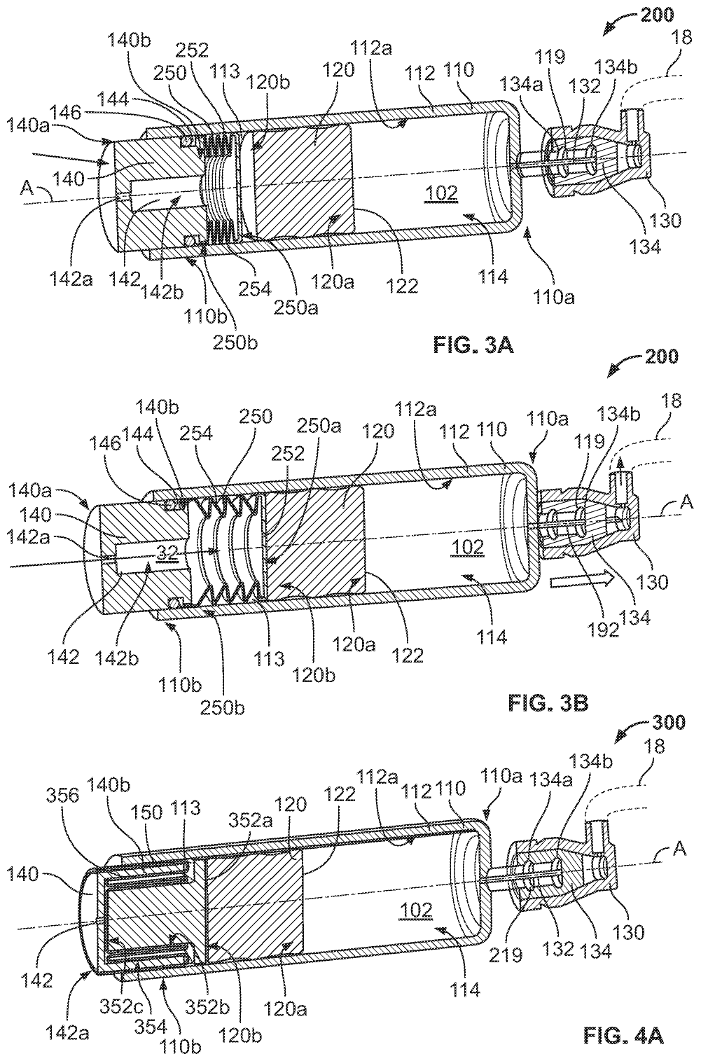

With reference to FIGS. 2A and 2B, the sealing arrangement 100 uses a first example seal member 150 in the form of a telescoping bellows. As previously noted, the second end 150b of the seal member 150 is operably coupled and sealed to the plug 140. The first end 150a includes a coupling portion 152 in the form of a generally flat region that contacts the second end 120b of the plunger 120. The first end 150a of the seal member 150 has a generally smaller dimension (e.g., a diameter or thickness) than the second end 150b of the seal member 150. The seal member 150 in this arrangement includes a number of folded sections 154 that, as illustrated in FIG. 2B, are generally aligned parallel to the longitudinal axis A and that expand in a telescoping manner during drug delivery.

More specifically, as previously noted, upon engaging the actuator 16, the activation mechanism 30 may release and/or urge the drive fluid 32. This force exerted by the drive fluid 32 on the seal member 150 causes the seal member 150 to expand and move towards the first end 110a of the container, which in turn causes the plunger 120 to move towards the first end 110a of the container 110. In these examples, the force from the drive fluid 32 may be sufficient to cause the seal member 150 to contact the plunger 120 and subsequently shift the container 110 relative to the plug 140. This relative movement of the container 110 causes the container needle 132 to pierce the septum 134, and thus create a flow path between the reservoir 114 and the fluid flow path 19. The seal member 150 will continue to push against the plunger 120 until the interior surface 122 of the plunger reaches the first end 110a of the container 110, upon which the drug 102 will be expelled from the container 110. In other examples, the plunger 120 exerts an urging force on the drug 102, which in turn may force the container needle 132 to pierce the septum 134.

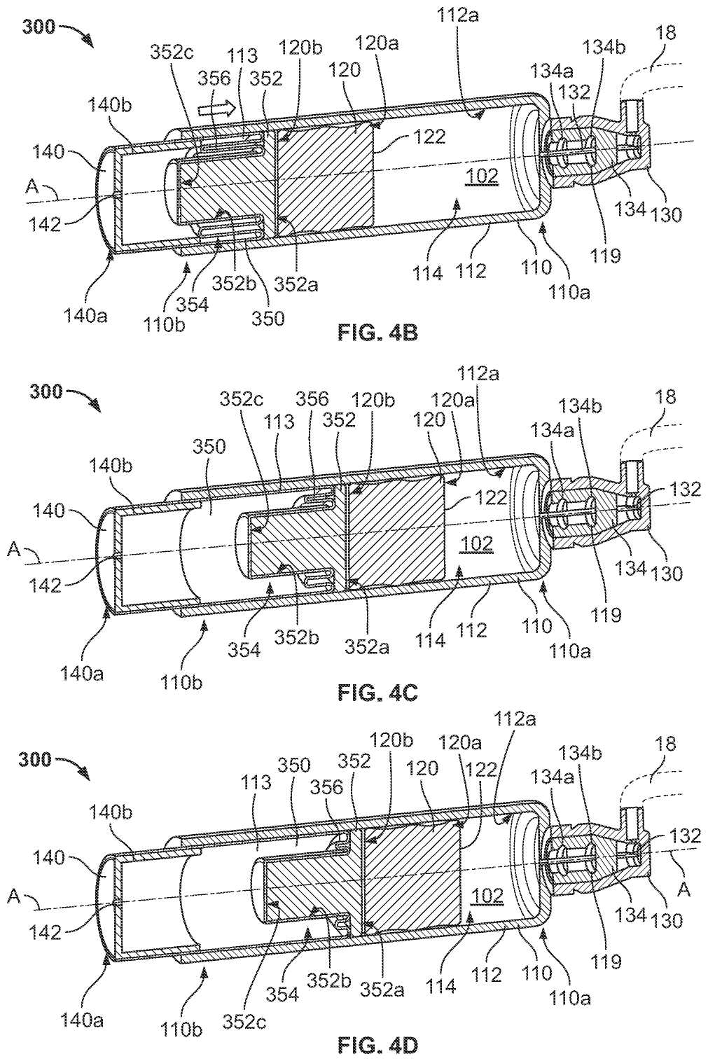

FIGS. 3A and 3B illustrate a second sealing arrangement 200 using a second example seal member 250 corresponding to the seal member 150 illustrated in FIGS. 2A and 2B, and as such, includes similar features and operation of the seal member 150 that will not be discussed in substantial detail. In the illustrated example, the seal member 250 is in the form of an accordion bellows. As before, the second end 250b of the seal member 250 is operably coupled and sealed to the plug 140. The first end 250a includes a coupling portion 252 in the form of a flat surface that contacts the second end 120b of the plunger 120. The seal member 250 in this arrangement includes a number of folded sections 254 that are generally aligned in a non-parallel, zig-zag direction relative to the longitudinal axis A and that expand in an accordion-like manner during drug delivery.

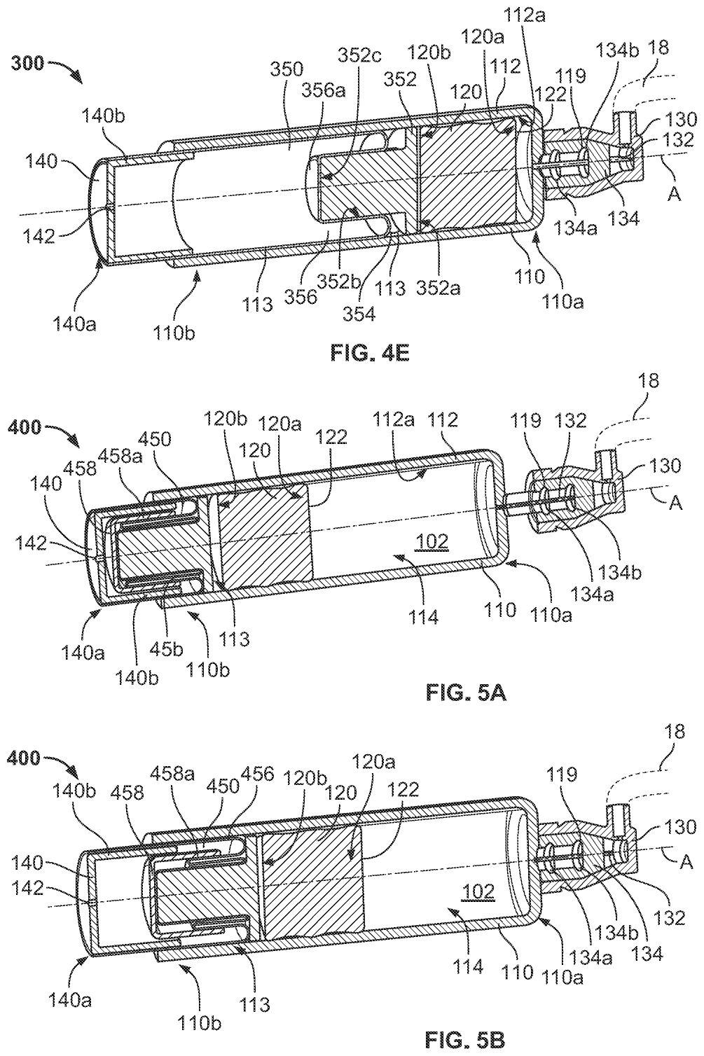

FIGS. 4A-4E illustrate a third sealing arrangement 300 using a third example seal member 350 corresponding to the seal members 150 and 250 illustrated in FIGS. 2A-3B, and as such, includes similar features and operation of the seal members 150, 250 that will not be discussed in substantial detail. In the illustrated example, the seal member 350 is in the form of a platform member 352 having a first contact surface 352a positioned adjacent to the second end 120b of the plunger 120. The plug 140 is in the form of a generally hollow, cup-like structure that has a sidewall 140b extending into the interior volume 113 of the container 110.

The platform member 352 further has an outer surface 352b that defines a rolling gap 354 with the interior surface 112a of the wall 112 of the container 110 (and/or the plug 140) and a second contact surface 352c. A flexible membrane 356 is operably coupled to the platform member 352 and has a storage state (as illustrated in FIG. 4A) and an operative state (as illustrated in FIGS. 4B-4E). In the illustrated example, the groove 144 and corresponding O-ring 146 are eliminated, and the flexible membrane 356 may be operably coupled to any portion of the plug (e.g., the first end 140a thereof) using any number of suitable approaches.

In the storage state, the flexible membrane 356 is folded at least partially within the rolling gap 354 and is thus aligned generally parallel to the longitudinal axis A. In the operative state, the drive fluid 32 causes the flexible membrane 356 to progressively unfold and slide against the outer surface 352b of the platform member 352 and/or the interior surface 112a of the wall 112 of the container 110. The urging force from the drive fluid 32 causes the flexible membrane 356 to push on the platform member 352, which urges the plunger 120 through the container 110. As illustrated in FIG. 4E, when the flexible membrane 356 is unfolded, an end 356a of the flexible membrane 356 continues to push against the second contact surface 352c of the platform member 352, which continues to urge the plunger 120 through the container 110, thereby expelling the drug 102.

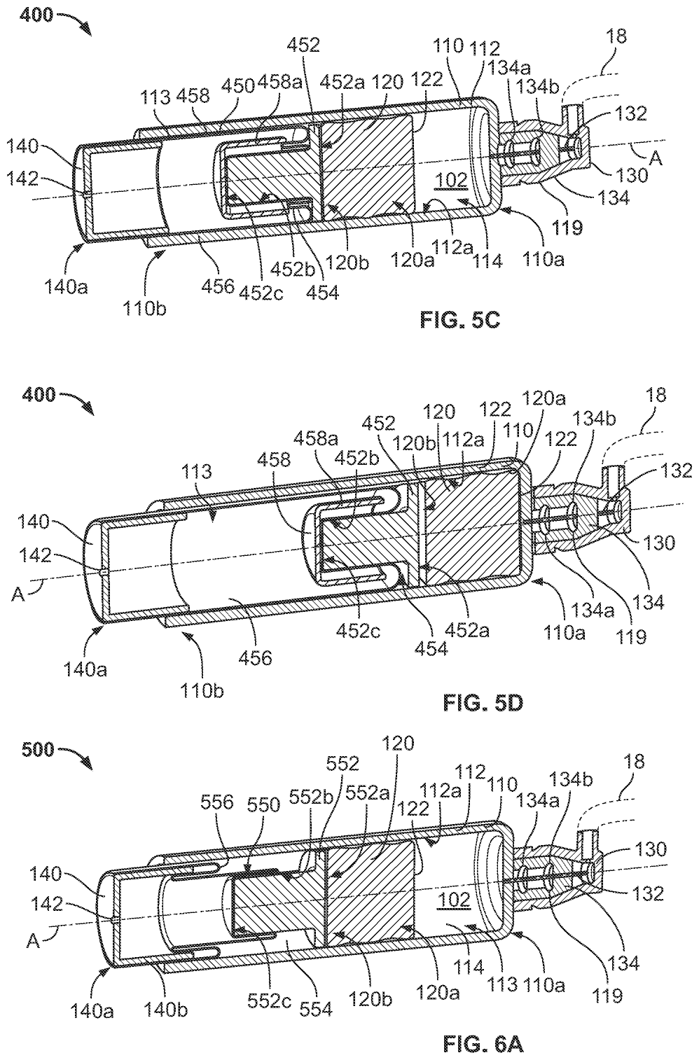

Turning to FIGS. 5A-5D, a fourth sealing arrangement 400 using a fourth example seal member 450 corresponding to the previous seal members 150, 250 and 350 is illustrated. The seal member 450 is similar in arrangement as the seal member 350 illustrated in FIGS. 4A-4E and as such, includes elements having similar two and/or three-digit reference numeral suffixes. The seal member 450 additionally includes a retaining feature in the form of a retaining cup 458 disposed about the platform member 452. The retaining cup 458 includes a sidewall 458a that is inserted in the rolling gap 454 that is used to guide movement of the flexible membrane 456 in the operative state to ensure that it properly unfolds/unrolls to urge the platform member 452 (and thus the plunger 120) through the container 110 to expel the drug 102 while reducing and/or limiting frictional forces between the interior surface 112a of the wall 112 and the flexible membrane 456.

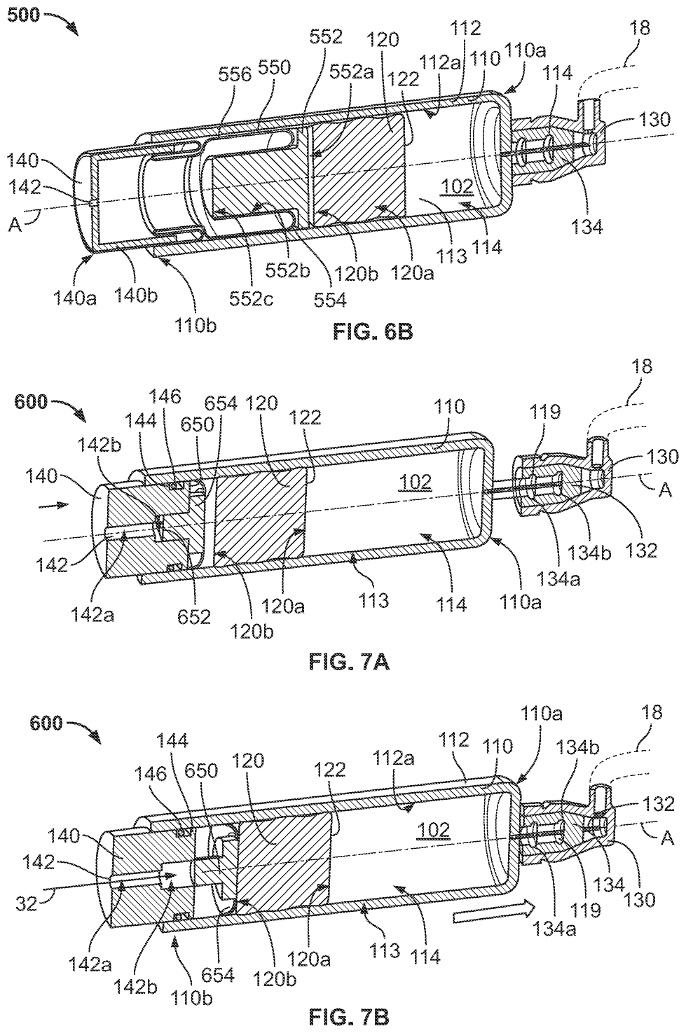

Turning to FIGS. 6A and 6B, a fifth sealing arrangement 500 using a fifth example seal member 550 corresponding to the previous seal members 150, 250, 350, and 450 is illustrated. More specifically, the seal member 550 is similar in arrangement as the seal member 350 illustrated in FIGS. 4A-4E and as such includes similar elements having similar two and/or three-digit reference numeral suffixes. The flexible seal member 550 differs from the flexible seal member 350 in that during the operational state, the flexible membrane 556 inflates outwardly in a balloon-like manner to abut the interior surface 112 of the wall 112 of the container 110 during drug administration to urge the plunger 120.

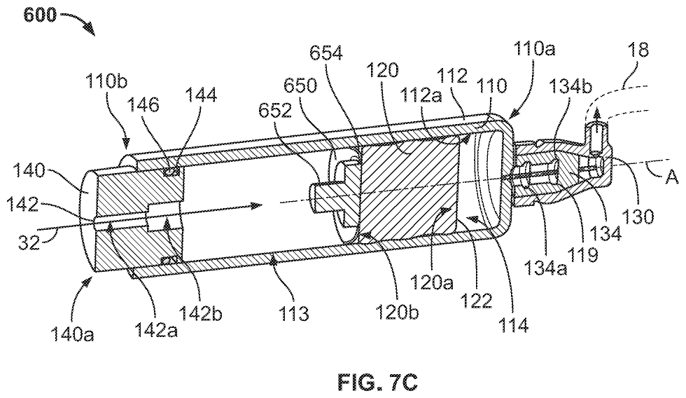

Turning to FIGS. 7A-7C, a sixth sealing arrangement 600 using a sixth example seal member 650 corresponding to the previous seal members 150, 250, 350, 450, and 550 is illustrated, and as such, includes similar features and operation of the seal member 150 that will not be discussed in substantial detail. In the illustrated example, the seal member 650 is in the form of a cup seal member that itself resembles a plunger. The seal member 650 includes a narrow portion 652 and a facing end 654. In the illustrated example, the narrow portion 652 is initially disposed in the wide region 142b of the bore 142, and prior to drug delivery, the facing end 654 is spaced away from the second end 120b of the plunger 120. In other examples, the facing end 654 may be initially configured to abut the second end 120b of the plunger 120.

As illustrated in FIG. 7B, upon activation of the drug delivery device 10, the drive fluid 32 flows through the bore 142 and urges the seal member 650, more specifically the facing end 654 thereof, to contact the plunger 120. As before, the container 110 then shifts to the right (in the illustrated example) and pushes the needle 132 through the septum 134, thus creating the drug delivery path. As illustrated in FIG. 7C, the drive fluid 32 continues to urge the seal member 650 and thus the plunger 120 through the interior volume 113 of the container 110 to expel the drug 102 while providing a secondary seal from the drive fluid 32.

So configured, the various embodiments of the seal member described herein inhibit and/or prevent the drive fluid 32 from entering into the reservoir 114, from contacting and/or potentially leaching into the plunger 120, and/or from contacting and potentially leaching into a wall of the container 110, thereby reducing potential damage to the drug 102. As a result, the composition of the drug 102 should not be altered by the drive fluid 32, ensuring the patient receives an uncompromised dose of the drug.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 146B7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); Vectibix® (panitumumab), Xgeva® (denosumab), Prolia® (denosumab), Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, or BPS 804 (Novartis) and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery device comprising:
a housing releasably coupled with a patient;
a container disposed in the housing and including a wall with an interior surface;
a plunger having an interior surface and being moveably disposed in the container, the interior surface of the wall of the container and the interior surface of the plunger defining a reservoir adapted to contain a drug;
an activation mechanism adapted to release a drive fluid for moving the plunger through the container to expel the drug from the container; and
a seal member moveably disposed in the container and adapted to inhibit ingress of the drive fluid into the reservoir.

2. The drug delivery device of claim 1, wherein upon engaging the activation mechanism, the drive fluid contacts the seal member to cause the plunger to move through the container to expel the drug from the container.

3. The drug delivery device of claim 1, wherein the drive fluid comprises at least one of a pressurized liquid or a pressurized gas.

4. The drug delivery device of claim 1, wherein the seal member is adapted to expand upon the release of the drive fluid, wherein the seal member optionally includes a first portion fixedly coupled with the plunger and a second portion fixedly coupled with the interior surface of the wall of the container.

5. (canceled)

6. The drug delivery device of claim 1, wherein the seal member comprises (a) a telescoping bellows operably coupled to the plunger, or (b) an accordion bellows operably coupled to the plunger.

7. (canceled)

8. The drug delivery device of claim 1, wherein the movable seal member comprises:
a platform member with an outer surface defining a rolling gap with the interior surface of the wall of the container and the outer surface of the platform member; and
a flexible membrane operably coupled to the platform member, the flexible membrane having a storage state wherein the flexible membrane is folded at least partially within the rolling gap, and an operative state wherein the drive fluid causes the flexible membrane to unroll and slide against at least one of the platform member or the interior surface of the wall of the container.

9. The drug delivery device of claim 8, further comprising a retaining cup disposed about the platform member for guiding the flexible membrane in the operative state.

10. The drug delivery device of claim 1, further comprising a plug covering an opening formed in the wall of the container.

11. The drug delivery device of claim 10, wherein the plug includes (a) a drive fluid channel to allow the drive fluid to flow therethrough upon being released, or (b) a coupling region that accommodates a portion of the seal member and the coupling region optionally includes a groove to accommodate a portion of the seal member and an O-ring.

12. (canceled)

13. (canceled)

14. The drug delivery device of claim 1, further comprising:
(a) a needle and a sterile barrier moveable relative to each other, a tip of the needle initially being exterior to or embedded within the sterile barrier, wherein optionally, upon engaging the activation mechanism, relative movement between the needle and the sterile barrier causes the needle to pierce through the sterile barrier to allow the drug to be expelled from the container, or
(b) an insertion mechanism configured to be operably connected in fluid communication with the container, the insertion mechanism including at least one of a needle or a cannula to be inserted into the patient for delivering the drug to the patient.

15. (canceled)

16. (canceled)

17. An assembly for a drug delivery device, the assembly comprising:
a container including a wall with an interior surface;
a plunger having an interior surface and being moveably disposed in the container, the interior surface of the wall of the container and the interior surface of the plunger defining a reservoir adapted to contain a drug;
an activation mechanism adapted to release a drive fluid for moving the plunger through the container to expel the drug from the container; and
a seal member moveably disposed in the container and adapted to inhibit ingress of the drive fluid into the reservoir.

18. The assembly of claim 17, wherein upon engaging the activation mechanism, the drive fluid contacts the seal member to cause the plunger to move through the container to expel the drug from the container.

19. The assembly of claim 17, further comprising:
a housing releasably coupled with a patient, the housing at least partially accommodating the drive assembly; and
a needle and a sterile barrier moveable relative to each other, a tip of the needle initially being exterior to or embedded within the sterile barrier.

20. The assembly of claim 19, wherein the housing is in the form of an autoinjector or a drug delivery device.

21. The assembly of claim 19, wherein the seal member is adapted to expand upon the release of the drive fluid, wherein the seal member optionally includes a first portion fixedly coupled with the plunger and a second portion fixedly coupled with the interior surface of the wall of the container.

22. (canceled)

23. The assembly of claim 17, wherein the seal member comprises a telescoping bellows operably coupled to the plunger, or (b) an accordion bellows operably coupled to the plunger.

24. (canceled)

25. The assembly of claim 17, wherein the seal member comprises:
a platform member with an outer surface defining a rolling gap with the interior surface of the wall of the container and the outer surface of the platform member; and
a flexible membrane operably coupled to the platform member, the flexible membrane having a storage state wherein the flexible membrane is folded at least partially within the rolling gap, and an operative state wherein the drive fluid causes the flexible membrane to unroll and slide against at least one of the platform member or the interior surface of the wall of the container.

26. The assembly of claim 25, further comprising a retaining cup disposed about the platform member and for guiding the flexible membrane in the operative state.

27. The assembly of claim 17, further comprising a plug covering an opening formed in the wall of the container.

28. The assembly of claim 27, wherein the plug includes (a) a drive fluid channel to allow the drive fluid to flow therethrough upon being released, or (b) a coupling region that accommodates a portion of the seal member and wherein the coupling region optionally includes a groove to accommodate a portion of the seal member and an O-ring.

29. (canceled)

30. (canceled)
